(19) United States
(12) Patent Application Publication
(10) Pub. No.: US 2004/0038950 A1
(43) Pub. Date: Feb. 26, 2004

(54) Pharmaceutical composition for treating IL-1 related diseases or disorders

(76) Inventors: Sang-Cheol Seong, Seoul (KR); Myung-Chul Lee, Seoul (KR); Hyun-Chul Jo, Seoul (KR); Jung-Sun Park, Seoul (KR); Mi-Young Jeong, Seoul (KR)

Correspondence Address: NIXON & VANDERHYE, PC, 1100 N GLEBE ROAD, 8TH FLOOR, ARLINGTON, VA, 22201-4714, US

(21) Appl. No.: 10/358,249

(22) Filed: Feb. 5, 2003

(30) Foreign Application Priority Data
Aug. 26, 2002 (KR) 2002-50568

(51) Int. Cl. A61K 31/57

(52) U.S. Cl. 514/170; 514/178

(57) ABSTRACT

The present invention relates to a pharmaceutical composition for treating (IL-1)-related disease or disorder, which comprises: (a) a therapeutically effective dose of dehyroepiandrosterone or its derivative represented by the formula (I); and (b) a pharmaceutically acceptable carrier:

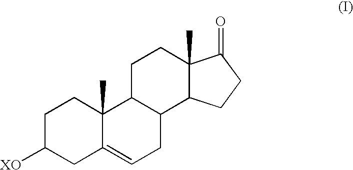

wherein X is H,

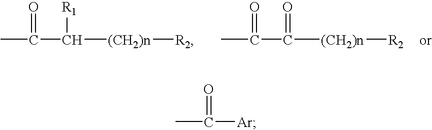

R₁ is H or —NH₂; R₂ is H, —COOH, —NH₂ or

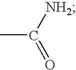

Ar is unsubstituted or substituted phenyl; and n is an integer of 1-20.

### BACKGROUND OF THE INVENTION

1. Field of the Invention

The present invention relates to a pharmaceutical composition and a method for treating (IL-1)-related disease or disorder, more particularly, to a pharmaceutical composition comprising dehyroepiandrosterone or its derivative as active ingredient and a method for treating (IL-1)-related disease or disorder.

2. Description of the Related Art

Interleukin-1 (hereinafter referred to as IL-1), one of immunoregulatory proteins, is produced in various cells such as monocyte and macrophage. IL-1 comprises IL-1α and IL-1β, which commonly exhibit diverse biological activities.

IL-1 has been reported to cause various diseases or disorders although it is a pivotal molecule in immunoregulatory reaction.

For example, IL-1 is involved in acute and chronic inflammation and autoimmune diseases. In rheumatoid arthritis, IL-1 causes inflammatory reaction in affected joint and destruction of cartilage proteoglycans. In addition, IL-1 has been implicated in various destructive bone diseases such as osteoarthritis and multiple myeloma (Bataille, R. et al., Int. J. Clin. Lab. Res., 21:283(1992)). Diseases associated with IL-1 include inflammatory diseases (e.g. osteoarthritis, pancreatitis and asthma), autoimmune diseases (e.g. glomerular nephritis, rheumatoid arthritis, scleroderma and alphosis), bone diseases (e.g. osteoporosis and multiple myeloma-related bone diseases), infectious diseases (e.g. septicemia and septic shock).

Investigators have discovered antagonists against IL-1 responsible for various diseases described above. For example, prostaglandin is known to inhibit the action of IL-1, and urine from febrile patients is reported to contain IL-1 inhibitor of 20-30 kD (Z. Liao, et al., J. Exp. Med., 159:126(1984)). Another example is a report by W. Arend et al.(J. Immun., 134:3868(1985)) that monocytes cultured on adherent immune complexes produce an IL-1 inhibitor.

Throughout this application, various patents and publications are referenced and citations are provided in parentheses. The disclosure of these patents and publications in their entities are hereby incorporated by references into this application in order to more fully describe this invention and the state of the art to which this invention pertains.

### SUMMARY OF THE INVENTION

The present invention describes that dehyroepiandrosterone and its derivatives can specifically inhibit IL-1 and thereby provide effective treatment of diseases or disorders caused by IL-1.

Accordingly, it is an object of this invention to provide a pharmaceutical composition for treating (IL-1)-related diseases or disorders.

It is another object of this invention to provide a method for treating (IL-1)-related disease or disorder.

Other objects and advantages of the present invention will become apparent from the detailed description to follow and together with the appended claims and drawings.

### BRIEF DESCRIPTION OF THE DRAWINGS

FIG. 1 is a graph showing the effects of various concentrations of DHEA on human chondrocyte proliferation by [³H]thymidine incorporation. Cells were collected after 1, 2, 3, and 7 days of incubation in the absence or presence of various concentrations (10, 50, and 100 μM) of DHEA.

FIG. 2 is a graph showing the effect of various concentrations of DHEA on glycosaminoglycan synthesis. Cells were collected after 1, 2, 3, and 7 days of incubation in the absence or presence of various concentrations (10, 50, and 100 μM) of DHEA.

FIGS. 3-6 represent the RT-PCR results showing the effects of DHEA on type I collagen, type II collagen, MMP-1, MMP-3, and TIMP-1 mRNA expression in chondrocyte. Human chondrocytes cultured in alginate bead were treated with DHEA for 72 hours or with 100 μM DHEA for various time periods. The intensity of the band is determined by densitometry.

FIG. 7 shows the results of Western blotting showing the effects of DHEA on MMPs-1 and 3, as well as on TIMP-1.

FIG. 8 shows the results of RT-PCR indicating the effects of IL-1β on mRNA expression of MMP-1 and MMP-3 in human chondrocytes. Chondrocyte alginate cultures were treated with 0, 10, 100, and 1000 μg/ml of IL-1β for 3 days. The PCR products were normalized with respect to the GAPDH band.

FIGS. 9-10 illustrate the effect of DHEA on (IL-1β)-induced MMP gene expression. Chondrocytes were incubated for 3 days with 0, 10, 50, and 100 μM DHEA in the presence of 1000 μg/ml IL-1β. mRNA Expression of MMP-1 and MMP-3 genes was determined by RT-PCR. The PCR products were normalized with respect to the GAPDH band.

FIG. 11 shows the results of RT-PCR experiments testing the effects of DHEA-pyruvate on mRNA expression of type II collagen, MMPs-1 and 3, and TIMP-1, respectively.

FIG. 12 shows the effects of DHEA on (IL-1)-induced gene expression of iNos, IL-6, and Cox-2.

FIG. 13 shows the effects of DHEA and DHEA-pyruvate on a knee joint of osteoarthritic rabbit model.

FIG. 14 shows the effect of DHEA+HA on a knee joint of osteoarthritic rabbit model.

### DETAILED DESCRIPTION OF THIS INVETNION

In one aspect of this invention, there is provided a pharmaceutical composition for treating (IL-1)-related disease or disorder, which comprises: (a) a therapeutically effective dose of dehyroepiandrosterone or its derivative represented by the formula (I); and (b) a pharmaceutically acceptable carrier:

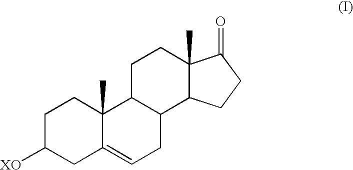

wherein X is H,

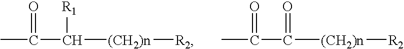

or

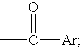

R₁ is H or —NH₂; R₂ is H, —COOH, —NH₂ or

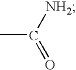

Ar is unsubstituted or substituted phenyl; and n is an integer of 1-20.

The present inventors have carried out an intensive research to develop a novel compound capable of inhibiting the interleukin-1 (IL-1) activity and found that dehyroepiandrosterone (hereinafter referred to as DHEA) and its derivatives can inhibit IL-1 and produce clinically therapeutic effect on osteoarthritis which is instigated by IL-1.

According to a preferred embodiment, the present compound, showing more effective therapeutic effect on (IL-1)-related diseases or disorders, is depicted in the formula (I); in which X is H, pyruvate, aspartate, aspargine, butyrate, palmitate, unsubstituted benzoate or substituted benzoate (e. g. acetoxybenzoate and hydroxybenzoate).

More preferably, X is H, pyruvate, aspartate or aspargine, most preferably, H or pyruvate in the formula (I).

Meanwhile, conventional chemotherapies with DHEA may generate adverse side effects such as the enlargement of liver and gonad, which is ascribed to oxidative stress. To overcome the shortcomings, the present inventors have synthesized appropriate derivatives of DHEA (WO 00/05242). The more preferable derivatives of this invention, in which X represents H, pyruvate, aspartate or aspargine, significantly reduce the adverse side-effects described above while maintaining the desired therapeutic effect on (IL-1)-related diseases or disorders.

The (IL-1)-related diseases or disorders which can be treated with the present composition include (a) inflammatory diseases such as osteoarthritis, pancreatitis and asthma; (b) autoimmune diseases such as glomerular nephritis, rheumatoid arthritis, scleroderma and alphosis; and (c) infectious diseases such as septicemia and septic shock.

Most preferably, the pharmaceutical composition of this invention shows excellent therapeutic effect on osteoarthritis. When the pharmaceutical composition of this invention is used to the treat osteoarthritis, it down-regulates the production of matrix metalloproteinase (hereinafter referred to as MMP). This process was found to be mediated by IL-1, in particular MMP-1 or MMP-3, most advantageously, MMP-1. At the same time, the pharmaceutical composition of this invention up-regulates the production of type II collagen and tissue inhibitor of metalloproteinase (hereinafter referred to as TIMP), most advantageously, TIMP-1. The inventors have established that the pharmaceutical composition of this invention exerts its therapeutic effect on osteoarthritis by way of the above mechanism.

It is to be noted that the compound showing therapeutic effect on osteoarthritis by enhancing TIMP production has not yet been reported. Therefore, in treating osteoarthritis the pharmaceutical composition of this invention is judged novel because of the enhancement of TIMP production.

It is preferable that the pharmaceutical composition of this invention further comprises a hyaluronic acid (hereinafter referred to as HA) for the treatment of osteoarthritis. Since HA decreases the level of MMP-3, the pharmaceutical composition of this invention, containing DHEA or its derivative, can exhibit a synergic therapeutic effect to relieve osteoarthritis.

In the pharmaceutical compositions of this invention, the pharmaceutically acceptable carrier may be conventional one for formulation, such as lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methylhydroxy benzoate, propylhydroxy benzoate, talc, stearic acid, magnesium and mineral oil, but not limited thereto. Additionally, the pharmaceutical compositions of this invention may contain wetting agent, sweetening agent, emulsifying agent, suspending agent, preservatives, flavors, perfumes, lubricating agent, or mixtures of these substances.

The pharmaceutical compositions of this invention may be administered orally or parenterally, and the parenteral administration comprises intravenous injection, subcutaneous injection, intramuscular injection and intraarticular injection.

Furthermore, it is important to note that the pharmaceutical composition of this invention is effective only on osteoarthritis when it is intraarticuarly administered into the affected joint. For example, the pharmaceutical composition of this invention did not produce therapeutic effect on arthritis when it was administered topically.

The correct dosage of the pharmaceutical compositions of the invention will vary depending on the particular formulation, the mode of application, age, body weight and gender of patient, diet, disease status of patient, conjunctive drugs and adverse reactions. It is understood that the ordinary skilled physician will readily be able to determine and prescribe a correct dosage of this pharmaceutical compositions. Preferably, the daily dosage of this pharmaceutical compositions ranges from 0.001-100 mg per kg body weight.

According to the conventional techniques known to those skilled in the art, the pharmaceutical compositions of this invention can be formulated with pharmaceutical acceptable carrier and/or vehicle as described above, such as a unit dosage form. Non-limiting examples of the formulations include, but not limited to, a solution, a suspension or an emulsion, an extract, an elixir, a powder, a granule, a tablet, a capsule, emplastra, a liniment, a lotion and an ointment.

In another aspect of this invention, there is provided a method for treating IL-1 related disease or disorder, which comprises administering to a patient a pharmaceutical composition comprising (a) a therapeutically effective dose of dehyroepiandrosterone or its derivative represented by the formula (I); and (b) a pharmaceutically acceptable carrier:

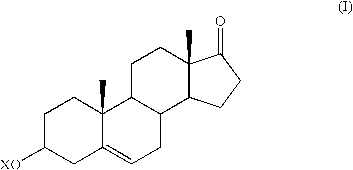

wherein X is H,

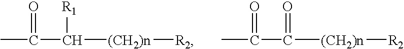

or

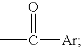

R₁ is H or —NH₂; R₂ is H, —COOH, —NH₂ or

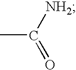

Ar is unsubstituted or substituted phenyl; and n is an integer of 1-20.

Since the present method uses this pharmaceutical composition described above, the common descriptions between them are omitted in order to avoid the complexity of this specification which may lead to undue multiplicity.

According to a preferred embodiment, the (IL-1)-related disease or disorder includes (a) inflammatory diseases such as osteoarthritis, pancreatitis and asthma; (b) autoimmune diseases such as glomerular nephritis, rheumatoid arthritis, scleroderma and alphosis; and (c) infectious diseases such as septicemia and septic shock. Most preferably, the (IL-1)-related disease or disorder is osteoarthritis.

When the present method is carried out to treat osteoarthritis, the production of MMP (in particular MMP-1 or MMP-3) is reduced. The most dramatic effect can be found with MMP-1. At the same time, the method of this invention up-regulates the production of type II collagen and TIMP, most notably, TIMP-1. By way of the molecular mechanism described earlier, the present method accomplishes its therapeutic effect on osteoarthritis.

It is preferable that the pharmaceutical composition used in the method of this invention further comprises HA when applied for a treatment of osteoarthritis.

Administration of the pharmaceutical composition in the present method may be performed orally or parenterally, and the parenteral administration comprises intravenous injection, subcutaneous injection, intramuscular injection and intraarticular injection. Importantly, the present method exerts therapeutic effect only on osteoarthritis when the administration is performed in a manner of the intraarticular injection.

The following specific examples are intended to be illustrative of the invention and should not be construed as limiting the scope of the invention as defined by appended claims.

### PREPARATION EXAMPLES

### Example I

### Synthesis of DHEA-Pyruvate

In anhydrous methylene chloride (250 ml) were dissolved Dehydroepiandrosterone (5.0 g) and dimethylaminopyridine (3.15 g). Separately pyruvic acid (3.0 g, 2.0 eq) was dissolved in a small quantity of methylene chloride and then added to the solution. Into the mixture was slowly injected a solution of dicyclohexylcarbodiimide (12.5 g) over 30 min at room temperature with the aid of a syringe. After stirring for 2 hours at room temperature, the solid formed was filtered off. The liquid filtrate was extracted with a mixture of water and ethyl acetate to separate an organic layer that was then dried over magnesium sulfate and removed of the solvent to give a colorless oil. Recrystallization allowed the production of the title compound as a white solid (yield: 75%): ¹H NMR spectrum (200 MHz, DMSO-d₆): 0.5-2.7(m, 28H), 4.4(m, 1H, CH), 5.4(d, 1H, ═CH)

### Example II

### Synthesis of DHEA-L-Aspartate

Step A. Synthesis of L-Aspartic Acid-Allylester Hydrochloride

In allyl alcohol (300 ml) was dissolved L-aspartic acid (8 g, 0.06 moles) and the solution was slowly added with trimethylsilyl chloride (19 ml, 0.15 moles) and then, stirred for 16 hours. Subsequently, ethyl ether (2 liters) was poured into the reaction to produce precipitates. After being filtered, the precipitates were washed with ether and dried to obtain the title compound (9.5 g) as a white solid (yield: 76%): ¹H NMR spectrum (200 MHz, D₂O): 3.20(d, 2H, CH₂), 4.30(t, 1H, CH), 4.72(m, 2H, CH₂CH═CH₂), 5.40(m, 2H, CH₂CH═CH₂), 5.96(m, 1H, CH₂CH═CH₂)

Step B. Synthesis of N-(t-butoxycarbonyl)-L-aspartic Acid-Ally Ester

At room temperature, a solution of di-t-butylcarbonate (4.31 g, 0.0197 moles) in dioxane was mixed with a solution of L-aspartic acid-allyl ester chloride (3.45 g, 0.0346 moles) in water. To the mixture was added triethyl amine (4.82 ml, 0.0346 moles) over 2 hours with stirring. After being additionally stirred for 12 hours, the reaction was extracted with hexane (50 ml×2) and the aqueous layer obtained was adjusted to about pH 2 with 1 N hydrochloric acid, after which it was extracted twice with ethyl acetate (50 ml×2) and the organic layer generated was washed with brine. The organic layer was dried over magnesium sulfate and evaporated in a vacuum evaporator to afford a colorless oily liquid (3.84 g, yield: 85%): ¹H NMR spectrum (200 MHz, CDCl₃): 1.45(s, 9H, C(CH₃)₃), 3.0(dd, 2H, CH₂), 4.55(m, 3H, CH, CH₂CH═CH₂), 5.40(m, 2H, CH₂CH═CH₂), 5.6(d, 1H, CONH), 5.90(m, 1H, CH₂CH═CH₂), 8.2(br, 1H, COOH)

Step C. Synthesis of N-(t-butoxycarbonyl)-L-aspartic Acid-Ally Ester-DHEA

In methylene chloride were dissolved N-(t-butoxycarbonyl)-L-aspartic acid-ally ester (2.09 g, 7.65 mmol), dehydroepiandrosterone (2.16 g, 7.49 mmol), and dimethylaminopyridine (1.40 g, 11.47 mmol). The solution was slowly added with dicyclohexylcarbodimide (5.52 g, 26.77 mmol) and stirred at room temperature for 24 hours. After completion of the reaction, the solution was filtered. The filtrate was extracted with ethyl acetate, after which the organic layer was dried over magnesium sulfate and evaporated in a vacuum evaporator to afford an oil with turbid color, which was purified by column chromatography on silica gel to obtain a white solid (2.8 g, yield 67.3%): ¹H NMR spectrum (200 MHz, CDCl₃): 1.5-2.6(m, 34H), 2.90(dd, 2H, CH₂), 4.65(m, 3H, CH, CH₂CH═CH₂), 5.35(m, 3H, CH₂CH═CH₂, CONH), 5.92(m 1H,CH₂CH═CH₂)

Step D. Synthesis of DHEA-L-aspartate

In a 50% trifluoroacetic acid was dissolved N-(t-butoxycarbonyl)-L-asparticacid-allyester-dehydroepiandrosterone (1.5 g, 2.75 mmol), followed by stirring the solution for 2 hours. Removal of the solvent in a vacuum evaporator left a colorless liquid that was then dissolved in a mixture of ethyl acetate (30 ml) and water (30 ml) and adjusted to pH 2 with 1 N hydrochloric acid. After being extracted from the resulting mixture, the organic layer was washed with brine, dried over magnesium sulfate, and removed of the solvent in a vacuum evaporator to give a colorless liquid.

The colorless liquid was dissolved in methylene chloride and palladium tetrakistriphenylphosphine (50 mg) plus triphenylphsphine (50 mg) was then added to the solution while stirring for 10 min. Subsequently, the reaction was added with a solution of sodium 2-ethylhexanoate (457 mg, 0.00275 mol) in ethyl acetate. After stirring the resulting solution for 2 hours, ethyl ether was poured to induce precipitate, after which centrifugation was performed to yield a pale yellow solid. The solid obtained were washed with ethyl acetate and then with ethyl ether and dried in vacuum evaporator to give the title compound as a white solid (yield: 50%): ¹H NMR spectrum (200 MHz, CD₃OD): 0.5-2.2(m, 24H), 2.40(m, 2H, CH₂), 3.6(m, 1H, CH), 4.6(m, 1H, CH), 5.40(d, 1H, ═CH)

### Example III

### Synthesis of DHEA-L-Asparagine

Step A. Synthesis of N-(t-butoxycarbonyl)-L-asparagine

In water (50 ml) was dissolved L-asparagine (4.14 g, 31.33 mmol) and the solution was added with sodium hydroxide (1.25 g, 31.33 mmol) and stirred until the solution became clear. While the reaction temperature was maintained at 0° C., a solution of di-t-butyldicarbonate (8.20 g, 37.59 mmol) in dioxane was added dropwise to the reaction. Stirring continued to be proceeded for 4 hours with gradual elevation to room temperature. The resulting solution was deprived of dioxane by use of a vacuum evaporator and ethyl acetate (50 ml) was poured, followed by adjusting to pH 2 with a 1 N hydrochloric acid solution. The precipitates thus produced were washed with ether and dried to give the title compound as a white solid (6.3 g, yield: 86%): ¹H NMR spectrum (200 MHz, DMSO-d₆): 1.44(s, 9H, C(CH₃)₃), 2.54(m, 2H, CH₂), 4.29(m, 1H, CH), 6.8-7.5(m, 1H, CONH)

Step B. Synthesis of DHEA-L-asparagine

In dimethyl formamide (35 ml) were dissolved N-(t-butoxycarbonyl)-L-asparagine (3.5 g, 15.0 mmol) and dehydroepiandrosterone (3.9 g, 13.56 mmol) along with dimethyl aminopyridine (3.11 g, 25.5 mmol), followed by slowly adding a solution of dicyclohexylcarbodiimide (10.8 g, 52.5 mmol) in dimethylformamide (10 ml) at room temperature. After the reaction was slowly stirred for 48 hours, the solid thus formed was filtered off and the dimethylformamide was removed from the liquid in a vacuum evaporator. The residual liquid was mixed with water and ethyl acetate to separate an organic layer that was then dried over magnesium sulfate, followed by removing the solvent in a vacuum to give a colorless liquid. This liquid is purified by column chromatography on silica gel to produce a white solid.

The solid was dissolved in a 50% trifluoroacetic acid solution, after which stirring was conducted for 2 hours. Following removal of the solvent from the solution, the resulting liquid was added with ethyl acetate and water and then, neutralized with a saturated sodium bicarbonate solution. The organic layer was separated, dried over magnesium sulfate, subjected to solvent-removal, and purified by column chromatography on silica gel to afford the title compound as a white solid (0.7 g, yield: 11.6%): ¹H NMR spectrum (200 MHz, DMSO-d₆): 0.5-2.4(m, 25H), 2.8(m, 2H, CH₂), 4.2(m, 1H, CH), 4.4(m, 1H, CH), 5.4(d, 1H, ═CH)

### Example IV

### Synthesis of DHEA-O-Benzoate

In dichloromethane (300 ml) were dissolved DHEA (2.9 g, 10.0 mmol) and benzoic acid (2.5 g, 20.0 mmol) and the solution was added with EDCI (3.8 g, 20.0 mmol) and DMAP (0.25 g, 2.0 mmol), followed by stirring for 24 hours at room temperature. After completion of the reaction, the solvents were removed under vacuum and the resultant was purified by column chromatography (dichloromethane:ethylacetate:hexane, 4:3:4) to yield the title compound as white powder (3.2 g, yield: 80%): ¹H NMR (400 MHz, CDCl₃): 7.99(d, 2H, J 7.8 Hz, ArH), 7.46(t, 1H, J=8.3 Hz, ArH), 7.36(t, 2H, J=8.3 Hz, ArH), 5.48(d, 1H, J=4.9 Hz), 4.75(m, 1H), 2.47(m, 3H), 2.44(t, J=7.3 Hz, 2H, CH₂), 2.14-1.18(m, 16H), 1.72(m, 2H, CH₂), 1.06(s, 3H, CH₃), 1.01(t, J=7.3 Hz, 3H, CH₃), 0.89(m, 3H, CH₃); ¹³C-NMR(100 MHz, CDCl₃) 220.97, 167.03, 139.29, 132.85, 130.56, 129.75, 129.72, 128.49, 128.41, 122.55, 76.48, 51.68, 50.29, 47.51, 37.67, 36.81, 36.72, 35.83, 31.43, 31.38, 30.78, 27.38, 21.87, 20.32, 19.30, 13.57

### Example V

### Synthesis of DHEA-O-(2-Hydroxy Benzoate)

The same procedures as Preparatory Example IV were performed to produce the title compound (yield: 78%) except that salicylic acid (2.8 g) was employed instead of benzoic acid: 1H NMR (400 MHz, CDCl₃): 7.69(d, 1H, J=7.8 Hz, ArH), 7.36(d, 1H, J=8.3 Hz, ArH), 7.12(t, 1H, J=8.3 Hz, ArH), 6.63(d, 1H, J=8.3 Hz, ArH), 5.45(d, 1H, J=4.9 Hz), 4.75(m, 1H), 2.47(m, 3H) 2.14-1.18(m, 16H), 1.06(s, 3H, CH₃), 0.89(m, 3H, CH₃); ¹³C NMR(100 MHz, CDCl₃): 220.93, 167.02, 156.54, 139.29, 133.36, 132.22, 122.55, 120.85, 117.43, 116.05, 76.56, 51.68, 50.29, 47.51, 37.67, 36.81, 36.72, 35.83, 31.43, 31.38, 30.78, 27.38, 21.87, 20.32, 19.30, 13.59

### Example VI

### Synthesis of DHEA-O-(2-Acetoxy-Benzoate)

The same procedures as Preparatory Example IV were performed to produce the title compound (yield: 82%) except that 2-acetoxy benzoic acid (3.6 g) was employed instead of benzoic acid: ¹H NMR (400 MHz, CDCl₃): 7.91(m, 1H ArH), 7.43(t, 1H, J=7.8, 7.3 Hz, ArH), 7.26(d, 1H, J=7.8 Hz, ArH), 6.99(t, 1H, J=7.8 Hz, ArH), 5.49(d, 1H, J=4.9 Hz), 4.75(m, 1H), 2.47(m, 3H), 2.24(s, 3H, COCH₃), 2.14-1.18(m, 16H), 1.06(s, 3H, CH₃), 0.89(m, 3H, CH₃); ¹³C NMR(100 MHz, CDCl₃): 220.95, 171.33, 168.80, 150.11, 139.29, 134.52, 131.16, 122.55, 122.32, 119.77, 118.87, 76.43, 51.68, 50.29, 47.51, 37.67, 36.81, 36.72, 35.83, 31.43, 31.38, 30.78, 27.38, 21.87, 20.32, 19.30, 16.64, 13.55

### Example VII

### Synthesis of DHEA-O-Butyrate

The same procedures as Preparatory Example IV were performed to produce the title compound (yield: 82%) except that butyric acid (1.8 g) was employed instead of benzoic acid: ¹H-NMR (400 MHz, CDCl₃): 5.38(d, 1H, J=4.9 Hz), 4.75(m, 1H), 2.47(m, 3H), 2.44(t, J=7.3 Hz, 2H, CH₂), 2.14-1.18(m, 16H), 1.72(m, 2H, CH₂), 1.06(s, 3H, CH₃), 1.01(t, J=7.3 Hz, 3H, CH₃), 0.89(m, 3H, CH₃); ¹³C-NMR(100 MHz, CDCl₃): 220.97, 172.00, 139.29, 122.55, 75.93, 51.68, 50.29, 47.51, 37.67, 36.81, 36.72, 36.02, 35.83, 31.43, 31.38, 30.78, 27.38, 21.87, 20.32, 19.30, 17.98, 13.54, 13.42

### Example VIII

### Synthesis of DHEA-O-Palmitate

The same procedures as Preparatory Example IV were performed to produce the title compound (yield: 89%) except that palmitic acid (5.2 g) was employed instead of benzoic acid: ¹H-NMR (400 MHz, CDCl₃): 5.37(d, 1H, J=4.9 Hz), 4.75(m, 1H), 2.47(m, 3H), 2.44(t, J=7.3 Hz, 2H, CH₂), 2.14-1.18(m, 18H), 1.72(m, 2H, CH₂) 1.26(bs, 24H, CH₂), 1.06(s, 3H, CH₃), 0.89(m, 3H, CH₃), 0.88(t, 3H, J=6.3 Hz, CH₃); ¹³C-NMR(100 MHz, CDCl₃): 220.99, 173.44, 139.29, 122.55, 75.31, 51.68, 50.29, 47.51, 45.93, 45.39, 45.36, 37.67, 36.81, 36.72, 35.83, 35.71, 31.88, 31.43, 31.38, 30.78, 29.69, 29.63, 29.57, 29.33, 29.24, 29.18, 28.24, 27.38, 24.92, 22.66, 21.87, 20.32, 19.30, 14.11, 13.56

### TEST EXAMPLES

### Example I

### Culture of Chondrocytes

Chondrocytes were isolated from the knee joint of patient undergoing a knee replacement due to osteoarthritis. Cartilage was digested with 0.2% protease Type XIV (Sigma) for 1 hour, followed by 3-hour incubation with 0.2% collagenase Type IA (Sigma) at 37° C. Following the incubation, the undigested cartilage fragments were removed using a 70 μm Nylon sieve. Chondrocytes were washed with DPBS and maintained in monolayer culture for 7 days in Dulbecco's modified Eagle's medium (DMEM; Life Technologies Inc.) which contained 10% fetal bovine serum (FBS; Life Technologies), 100 units/ml penicillin-streptomycin (Life Technologies) and 25 μg/ml L-ascorbic acid (Sigma). All experiments were performed with primary or first passage cells. The preparation of chondrocytes in alginate beads was carried out with 4×10⁶ cells per 1 ml of alginate. Alginate cultures were maintained for 7 days at 37° C., 5% CO₂ and 95% humidity and incubated with various concentrations of DHEA (0, 10, 50 or 100 μM) for a varing period of time. DHEA was purchased from Sigma and used after dissolving either in ethanol or dimethyl sulfoxide (DMSO). After a desired incubation period, cells were harvested by solubilizing the alginate beads in citrate buffer (55 mM sodium citrate, 0.15 M NaCl, pH 7.0) for 10 min at 37° C. and by centrifugation (300 g, 5 min).

### Example II

### Treatment with Recombinant Human IL-1β

Sixteen hours prior to a treatment, the complete culture medium was replaced with a serum-free medium. Chondrocyte alginate bead cultures were then treated with IL-1β (0, 10, 100 or 1000 μg/ml, (Calbiochem) and DHEA (0, 10, 50 or 100 μM) for 3 days.

### Example III

### Cell Proliferation, GAG Assay and Cellular DNA Content Measurement

For cell proliferation, [³H]thymidine assay was performed on a 96-well plate (Nunc) with one alginate bead per well. The cells were pulsed with 0.5 μCi [³H]thymidine per well for 16 hours. After collecting the cells using a cell harvester (Wallac), [³H]thymidine uptake was quantitated by measuring β emission on a β-counter (Wallac). To assay glycosaminoglycan (GAG) accumulation, alginate beads were incubated in complete DMEM medium with 10 μCi/ml radiolabeled sodium sulfate (Na₂³⁵SO₄) for 4 hours at 37° C. The cells were washed, resuspended and digested for 12 hours at 55° C. in papain buffer (200 μg/ml papain in 50 mM EDTA, 5 mM L-cysteine, pH 3.0). The ³⁵SO₄-labeled proteoglycan content was measured by a liquid scintillation counter (Wallac). The total cellular DNA was determined by Indole assay.

### Example IV

### RNA Extraction and Reverse Transcription-Polymerase Chain Reaction

Total RNA from chondrocytes was extracted using RNeasy Mini Kit (QIAGEN). Reverse transcription from 1 μg of total RNA was conducted using First Strand cDNA Synthesis Kit (MBI Fermentas) according to manufacturer's recommendation with random hexamers. The resulting cDNA was then amplified by polymerase chain reaction using AccuPower PCR Premix (Bioneer, Korea) in a final volume of 20 μl using three thermocycler temperatures (30 sec at 94° C., 30 sec at 60° C., and 30 sec at 94° C.). All reactions were determined to be in the linear range of amplification with the cycle number ranging from 14 to 30. The PCR products were analyzed by separation on a 2% agarose gel followed by ethidium bromide staining. Following electrophoresis, photographs were taken and analyzed using a densitometric program (TINA; Raytest Isotopenmeβgerate, Germany). Integrated density values for the genes in question were then normalized to the glyceraldehyde-3-phosphate dehydrogenase (GAPDH) values for a semiquantitative assessment. After normalizing with respect to GAPDH, percentage increase or decrease was determined. Each experiment was repeated at least three times. The sequences of PCR primers used were as follows: for GAPDH, 5′-ATTGTTGCCA TCAATGACCC-3′ and 5′-AGTAGAGGCAGGGATGATGTT-3′; human type I collagen, 5′-CTCGAGGTGGACACCACCCT-3′ and 5′-CAGCTGGATGGCCACATC GG-3′; human type II collagen, 5′-GAATTGGGTGTGGACATAGG-3′ and 5′-TACAGAGGTGTTTGACACAG-3′; human MMP-1, 5′-ATTCTACTGATATCGGGG CTTTGA-3′ and 5′-ATGTCCTTGGGGTATCCGTGTAG-3′; human MMP-3,5′-CTCACAGACCTGACTCGGTT-3′ and 5′-CACGCCTGAAGGAAGAGATG; human TIMP-1,5′-AATTCCGACCTCGTCATCAGG-3′ and 5′-ACTGGAAGCCCTTTTCA GAGC-3′.

### Example V

### Western Blot Analysis

To investigate the effect of DHEA on the protein concentration, Western blot analysis was performed. Alginate beads, which had been incubated with 0, 10 or 100μ of DHEA for three days, were dissolved as previously described, and the cell pellets were immediately lysed in lysing buffer containing 50 mM Tris (pH 8.0), 150 mM NaCl, 1 mM Na₃VO₄, 100 μg/ml phenylmethylsulfonyl fluoride (PMSF), 1 μg/ml aprotinin and 1% Triton X-100 (all from Sigma) and centrifuged at 16,000×g for 10 minutes. Protein concentration of the supernatant was determined by Bio-Rad protein assay (Bio-Rad, USA) using bovine serum albumin (BSA) as a standard. The synthesis of MMP-1, 3, and TIMP-1 was confirmed with 50 μg protein extract by Western blot analysis. In the Western blot experiments, goat anti-human antibodies specific to MMP-1, MMP-3 and TIMP-1 were used as the primary antibodies, respectively, and donkey anti-goat antibody conjugated with horseradish peroxidase (HRP) was employed as the secondary antibody. The immune complex band was developed with luminol as a chemiluminescent substrate for HRP enzyme.

### APPLICATION EXAMPLES

### Example I

### Osteoarthritis Induction and Intraarticular Injection of DHEA

Mature New Zealand White rabbits underwent bilateral anterior cruciate ligament transection (ACLT). ACLT was performed using a medial arthrotomy method. Postoperatively the animals were permitted cage (60 cm×60 cm×40 cm) activity without immobilization. The right knees received via intraartiticular injection DHEA, DHEA-pyruvate or DHEA+HA, 4 weeks after ACLT and once a week for 5 weeks. The left knees received 0.3 ml of intraarticular injection with control solution (carrier solution). After 9 weeks from ACLT, the animas were sacrificed.

### Example II

### RNA Extraction and Reverse Transcription-Polymerase Chain Reaction

Synovium (entire tissue) around the infrapatellar fat pads and cartilage tissue from the femoral condyle and the tibial plateau were harvested. The subsequent experimental procedure was the same as described in Test Example IV.

### Example III

### Topical Application of DHEA to Arthritic Patient

A. Subjects

Ten patients diagnosed to have degenerative arthritis were tested to investigate the therapeutic effect of DHEA in topical application. Testees had the symptoms including dolor of knee joint (resting or motion pain), swelling, deformation, tenderness, crepitation or stiffness of joint. Patients who had received intraarticular injection, steroid treatment or surgery within 1 month before this test were excluded, as well as those who had severe cardiac or liver diseases, kidney failure, gastrointestinal diseases, or poor systemic condition such as anomalotrophy. Besides, women who were pregnant or breast-feeding babies were excluded in this study.

B. Method

The volunteers were randomly grouped into Group I and Group II, and subject to the experiment for topical application of DHEA. A portion of knee joint showing dolor symptom was coated with either 5% DHEA or PBS (phosphate buffered saline) twice a day for 1 month. Any alteration of symptom was checked prior to the application, 1 week and then again 1 month during the application regimen.

i) Clinical Assessment

The severity of pain was scored ranging from 0 (most severe pain) to 20 points (no pain) at an interval of 5 points. The criteria were as follows: 20 points, no pain; 15 points, painful on rare occasions or painful when doing vigorous exercise or descending stairs; 10 points, taking analgesia on rare occasions, significantly painful when doing vigorous exercise but not interfered with everyday life or painful when ascending stairs; 5 points, required to take analgesia at all times, interfered with light exercise or incapable of descending stairs; and 0 point, experiencing severe pain at all times, interfered with everyday life or incapable of both ascending and descending stairs.

In addition, both joint effusion and medial joint-line tenderness were recorded during a physical test administered by a doctor based on the point rating scale: highness, 4; medium, 3; slightness, 2; and not found, 0.

The opinions on symptom were recorded as 5 categories relying on the subjective opinions of patients and the opinions of doctors regarding both dolor of patients and the results of physical tests: superiority, goodness, mediocrity, poorness and exceeding poorness.

In addition, the assessment regarding the level of improvement on symptoms was recorded in accordance with the following criteria: 3-step improvement, “exceeding improvement”; 2-step improvement, “improvement”; 1-step improvement, “slight improvement”; “no change”; and “aggravation”.

ii) Adverse Effects

Adverse effects were checked for the clinical test by appearance period, duration, severity and improvement.

Results

I. The Effect of DHEA Treatment on the Cell Proliferation and Proteoglycan Synthesis of Chondrocyte Cultures

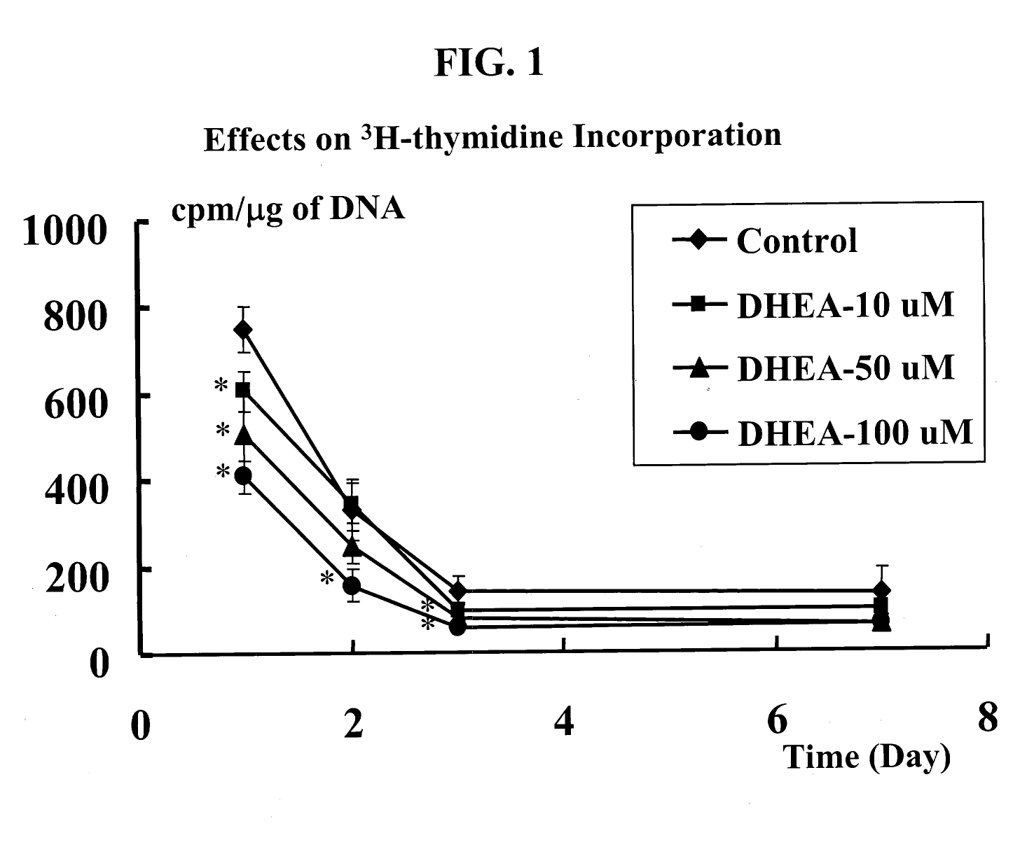

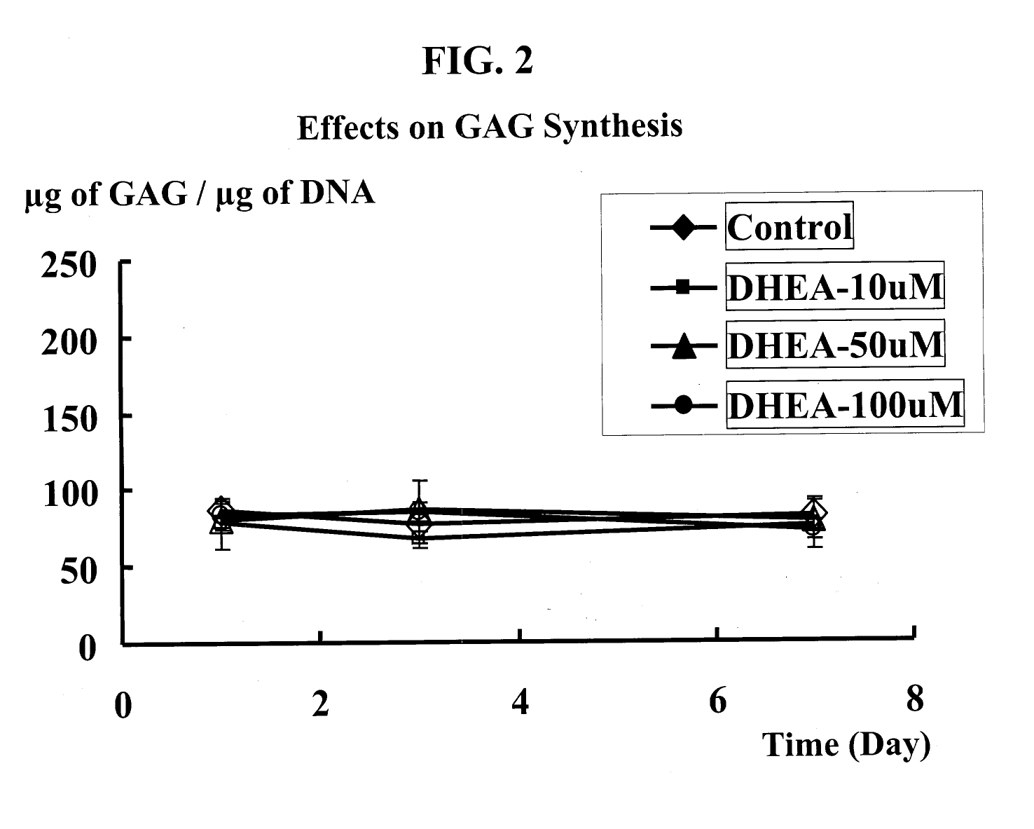

FIGS. 1 and 2 show that DHEA slightly reduced cell proliferation as early as day 3 of culture. The inhibition effect of DHEA on cell proliferation appears inversely proportional to DHEA concentration in the range tested (10-100 μM). The effect appeared to be insignificant after day 5.

The rate of proteoglycan synthesis remained unchanged when DHEA was present in the chondrocyte cultures during the same time period.

II. Effects of DHEA on the Production of Type I Collagen, Type II Collagen, MMP-1, MMP-3, and TIMP-1 mRNA

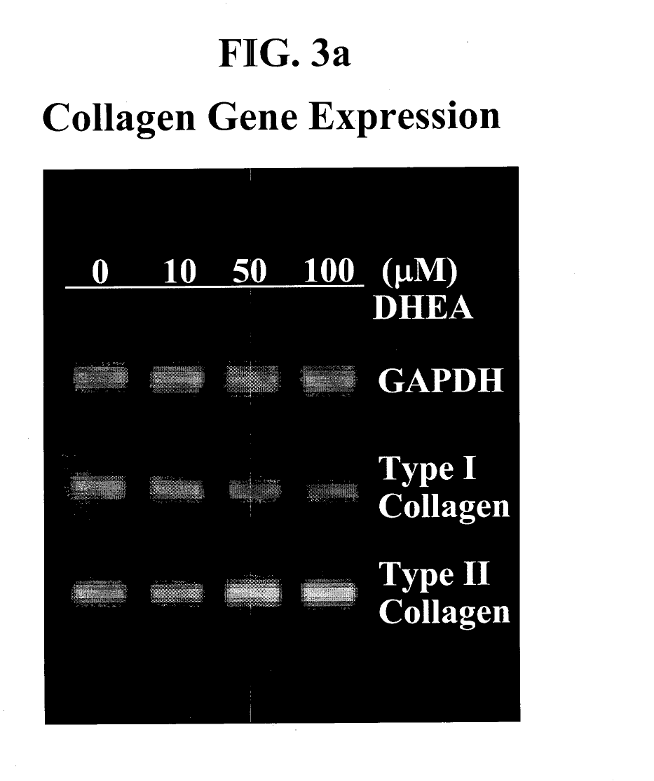

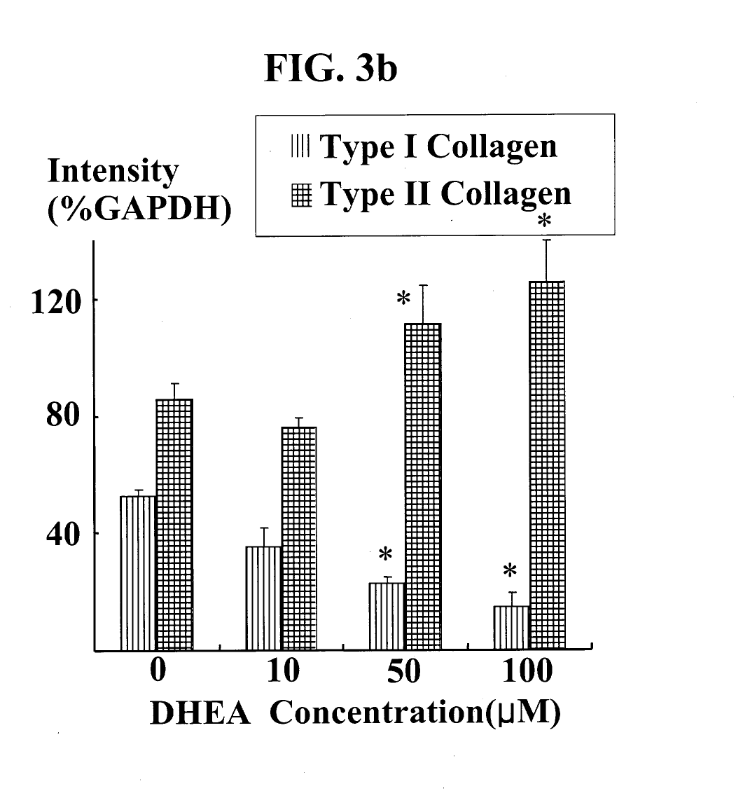

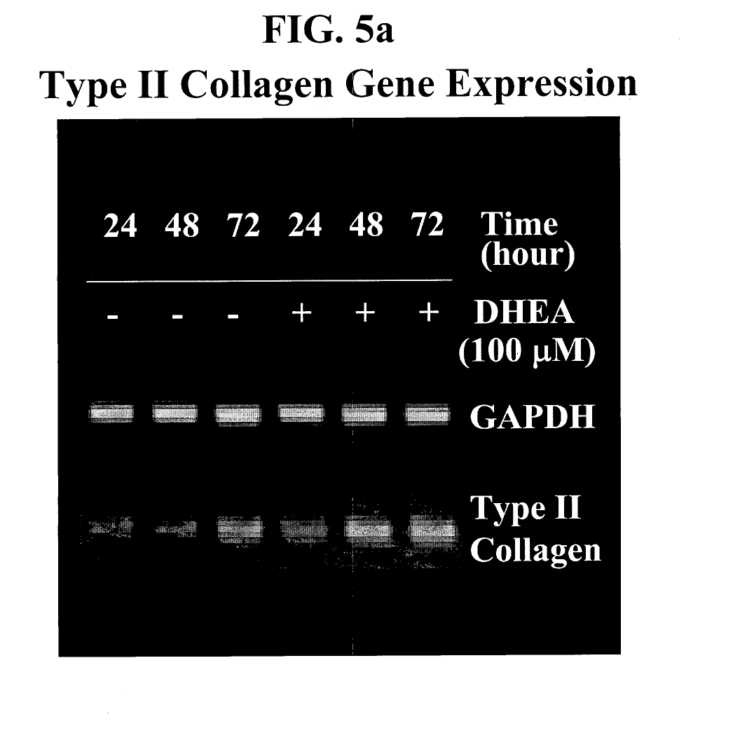

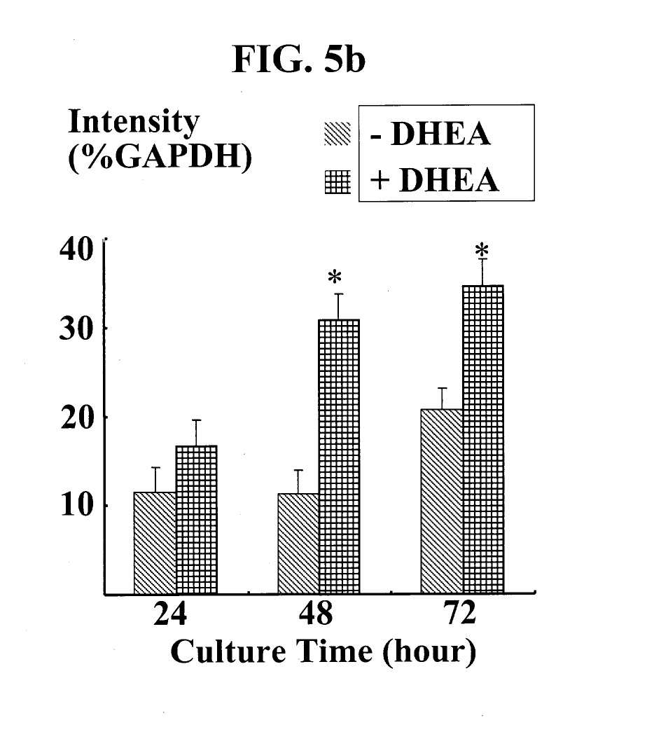

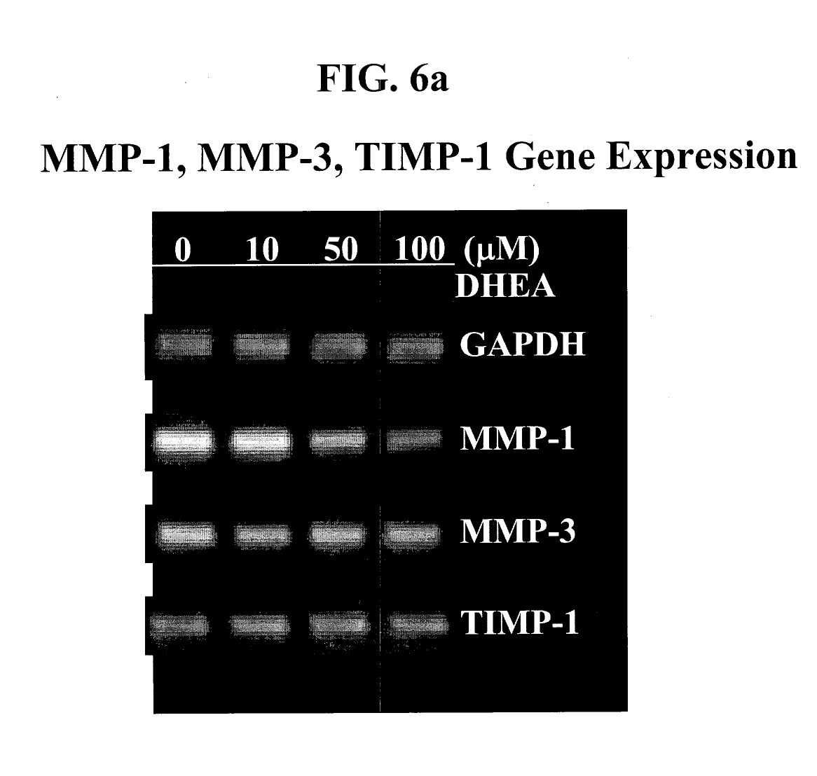

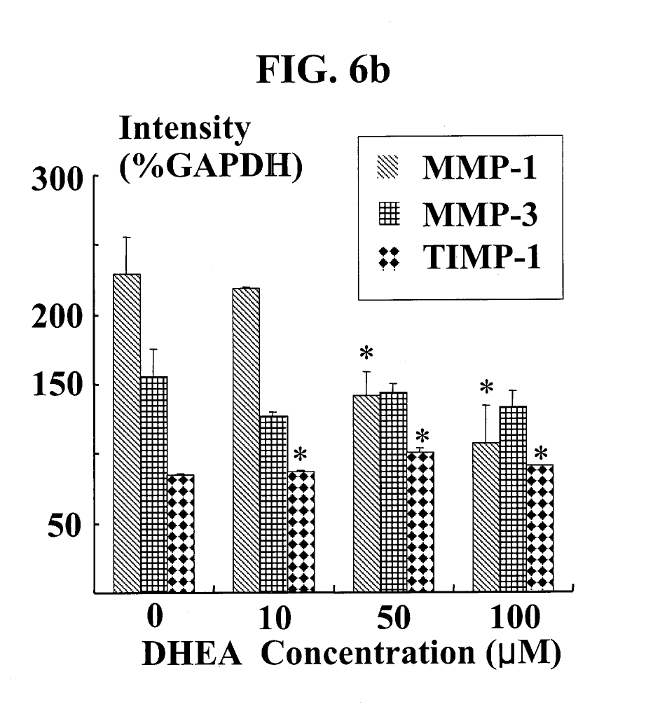

Chondrocytes were treated with various concentration of DHEA (10-100 μM) for 72 hours and mRNA levels of type I and II collagens, MMP-1 and -3, and TIMP-1 were determined by RT-PCR. DHEA treatment suppressed the production of type I collagen mRNA in a dose-dependent manner with maximal inhibition of 28% at 100 μM (p<0.001) (FIGS. 3-6). Up-regulation of type II collagen gene by DHEA was observed, showing 132% and 146% enhancement in the presence of 50 and 100 μM DHEA, respectively (p<0.01).

Since the stoicheometry of MMPs over TIMPs plays an important role in the formation or degradation of extracellular matrrix, the effect of DHEA concentration on the production of MMP-1, MMP-3, and TIMP-1 mRNAs was examined. DHEA treatment inhibited the level of MMP-1 in a dose-dependent manner up to 48% at 100 μM (p<0.01). DHEA slightly suppressed the production of MMP-3 but the inhibition (82-93%) was not dose-dependent. Expression of TIMP-1 mRNA was enhanced by DHEA, and the increase was statistically significant (p<0.05) and exhibited the maximum rate of 120% with 50 μM DHEA. Unexplicably, at DHEA concentration above 100 μM, the enhancement effect appears diminished.

Next, we investigated the time-dependent effect of DHEA on the expression of type I and II collagens, MMP-1 and -3, and TIMP-1 mRNAs in chondrocyte cultures.

When chondrocyte cultures were treated with 100 μM of DHEA for 24 h, 48 h, and 72 h, the expressions of type I collagen and MMP-1 mRNA were markly inhibited after 72 hr. Production of type I collagen was reduced by less than 50% after 24-72 hr of culture (p<0.05) while the level of MMP-1 mRNA was reduced by 43 and 35% after 24 and 48 hr, respectively (p<0.05). DHEA decreased expression of MMP-3 mRNA by 73% after 24 hr of culture (p<0.05), whereas, the inhibitory activity of DHEA on the MMP-3 increase by 131% after 72 hr. Incubation of chondrocytes with DHEA resulted in a marked increase in the level of type II collagen mRNA for 24-72 hr, with the maximal stimulation of 276% after 48 hr (p<0.05). Although the expression of TIMP-1 mRNA was unaffected by DHEA up to 48 hr, it exhibited an up-regulation of 129% (p<0.05) after 72 hr.

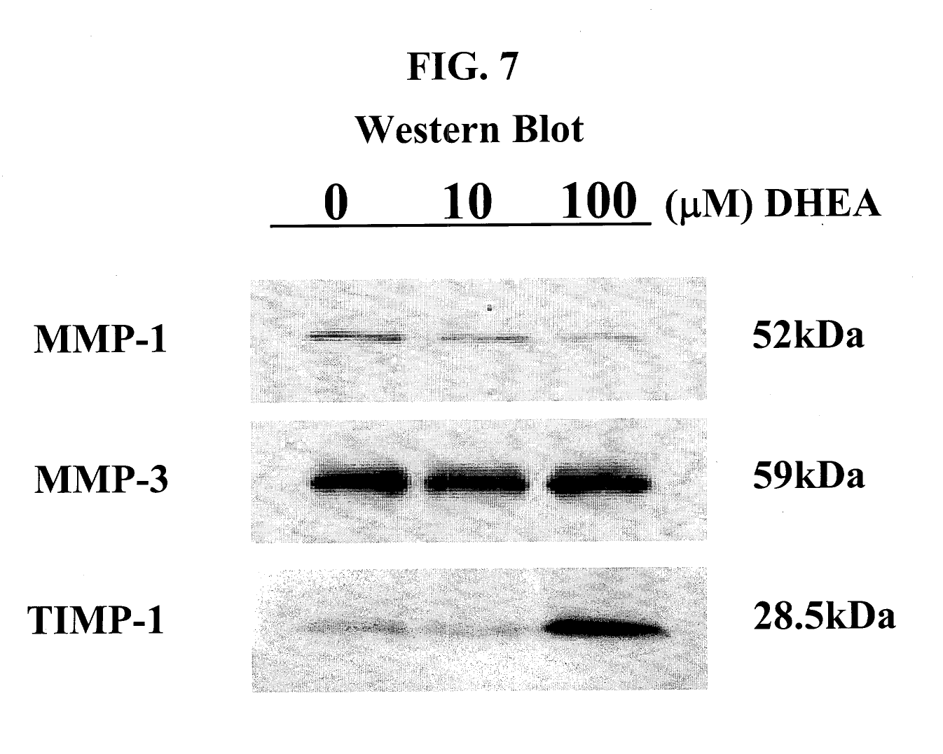

To determine whether the suppression of mRNA levels for MMPs-1 and 3 as well as the enhancement of that for TIMP-1 had been accompanied by an augmentation of protein synthesis, we evaluated the production of mature MMP-1 and MMP-3 and TIMP-1 in the lysates of chondrocytes by Western blot analysis (FIG. 7). The result indicates that MMP-1 level was reduced with increasing DHEA concentration while MMP-3 level did not change. The translational level of mature TIMP-1 increased with increasing concentration of DHEA. Western blot analysis revealed that transcriptional effect of DHEA might be associated with corresponding translational activity.

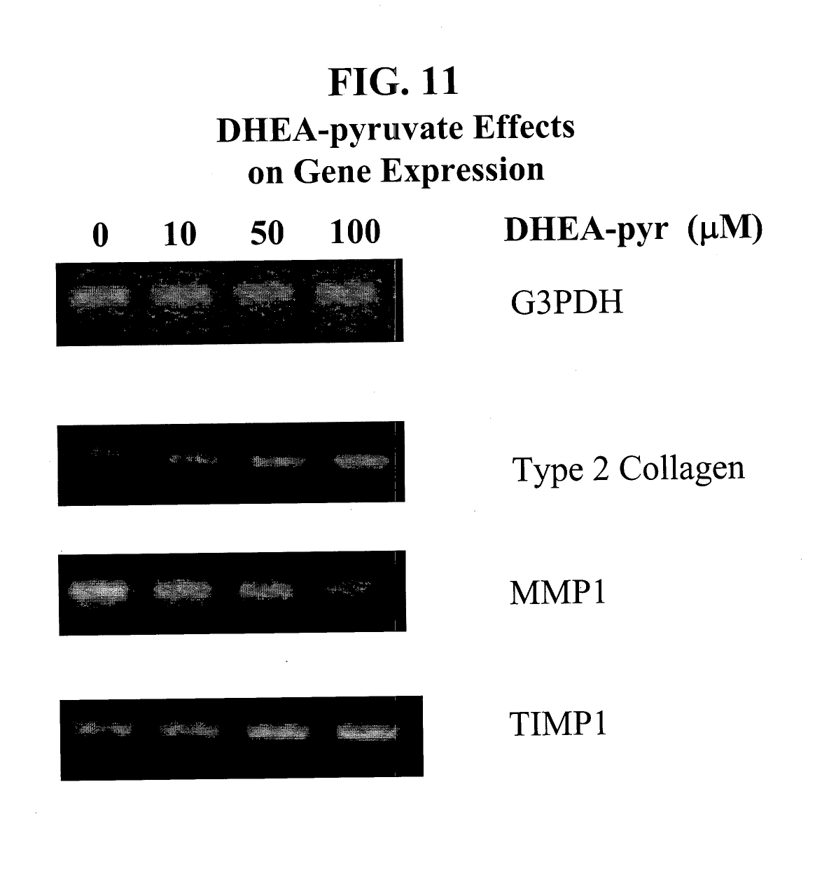

A similar phenomenon was evident when DHEA-pyruvate replaced DHEA (see FIG. 11).

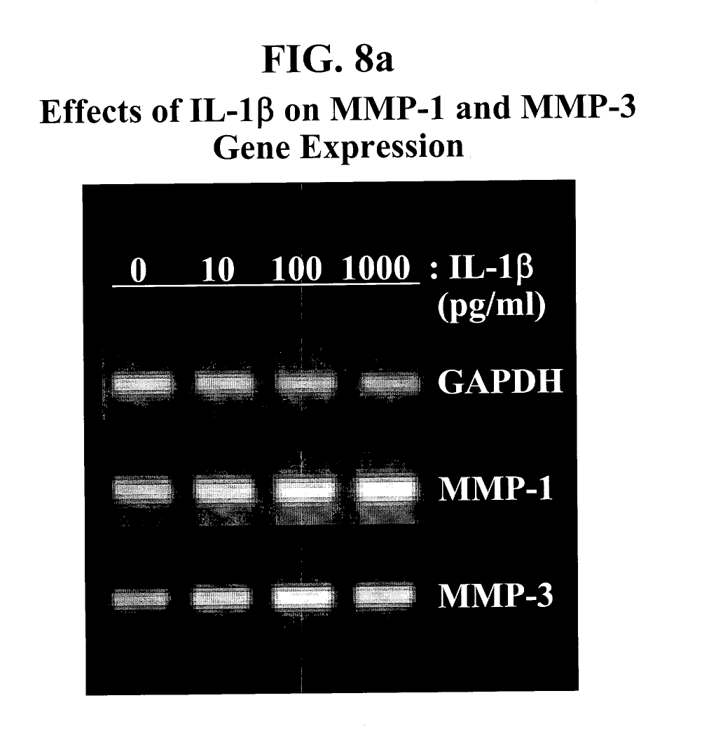

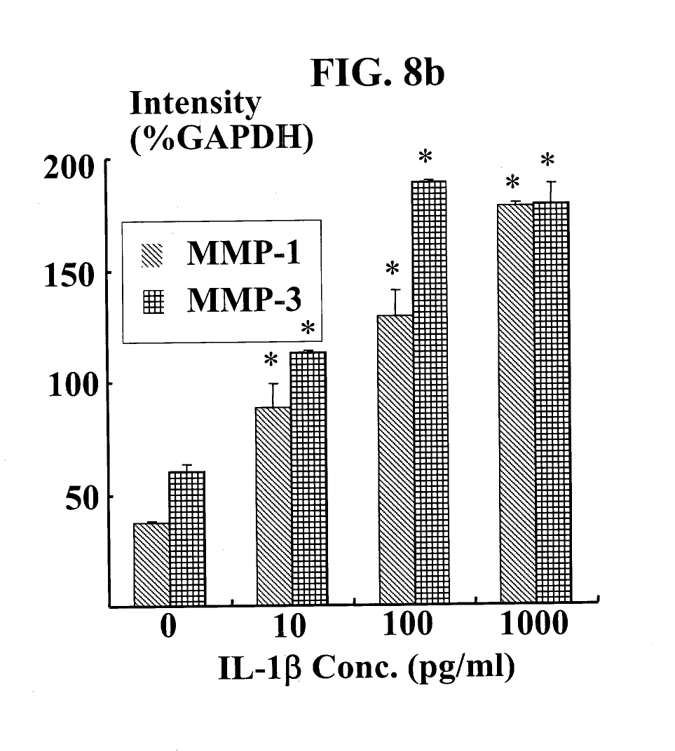

III. Effects of IL-1β on MMP-1 and MMP-3 mRNA Expressions in Chondrocyte Culture (FIG. 8)

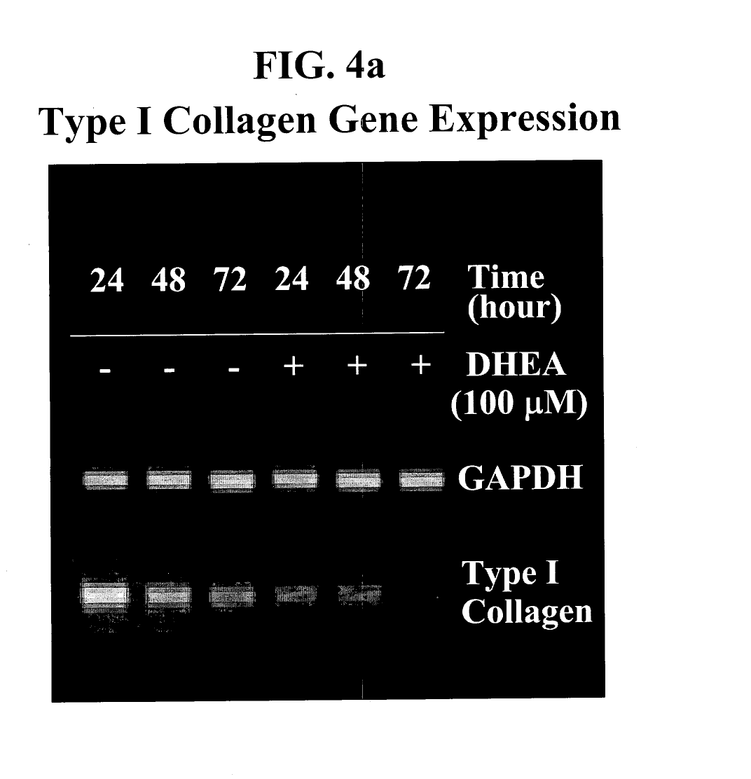

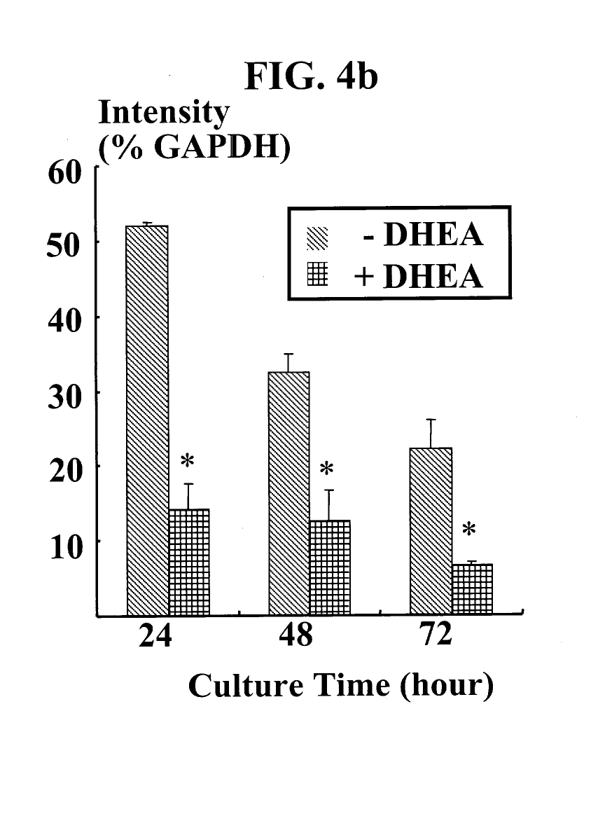

It was reported that elevated level of IL-1 stimulated the biosynthesis of proteolytic enzymes such as MMP-1 and MMP-3 from articular cartilage. To verify that IL-1 can induce production of MMP mRNAs, chondrocyte cultures were treated with an increasing amount of IL-1β (10, 100, and 1000 μg/ml) and the expressions of MMP-1 and MMP-3 mRNAs were assessed by RT-PCR as shown in FIG. 4. IL-1 β induced the production of MMP-1 as well as MMP-3 mRNAs in a dose-dependent manner. 1000 μg/ml of IL-1β was able to increase MMP-1 mRNA by as much as 470%, while 100 μg/ml of IL-1β increased MMP-3 mRNA by 310%.

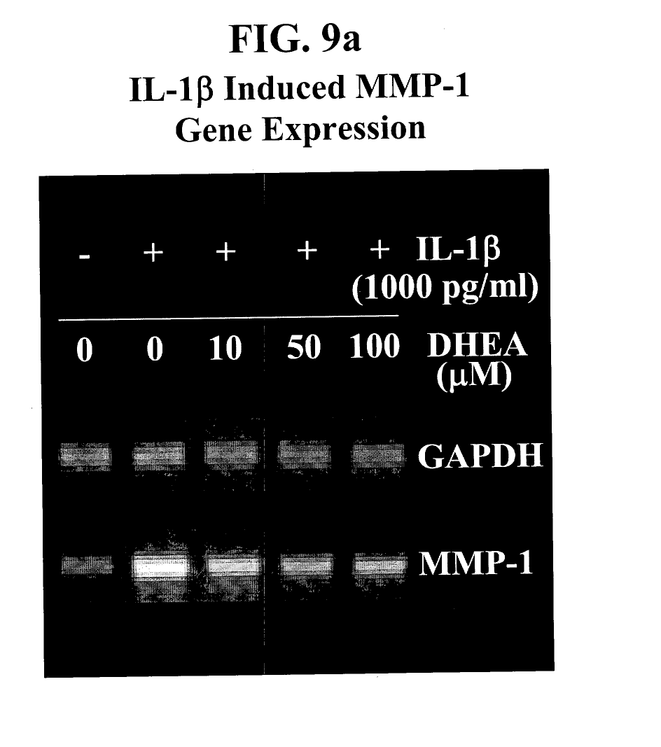

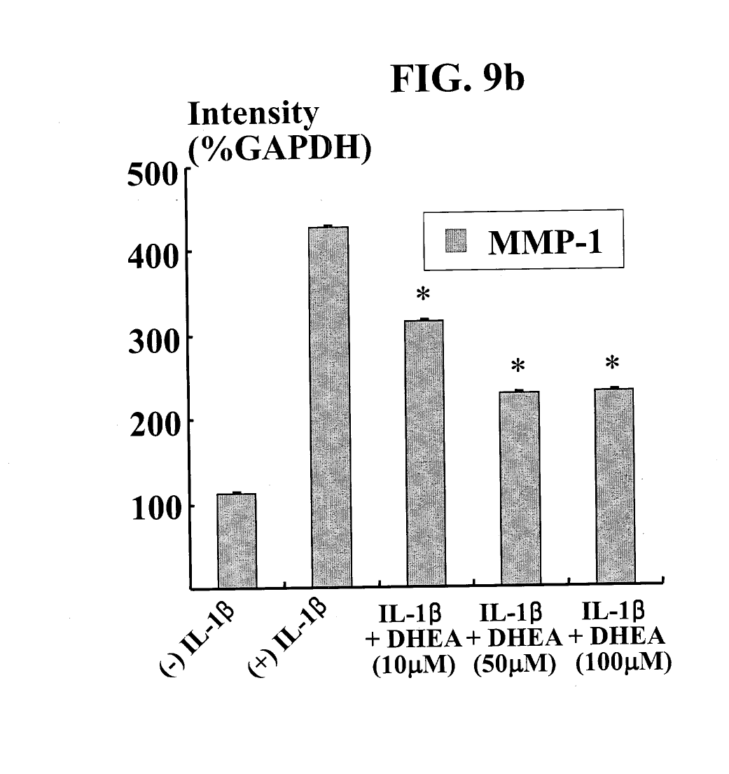

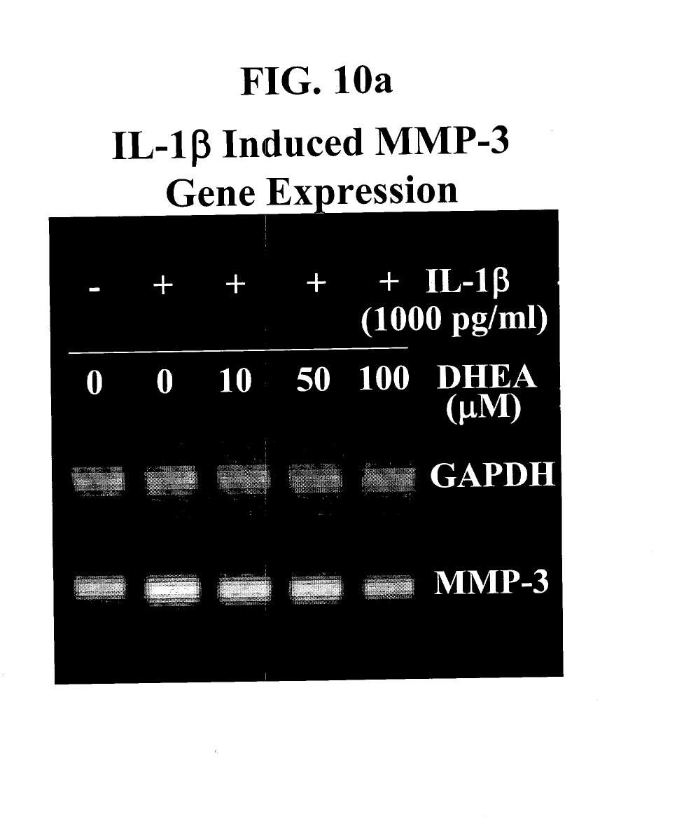

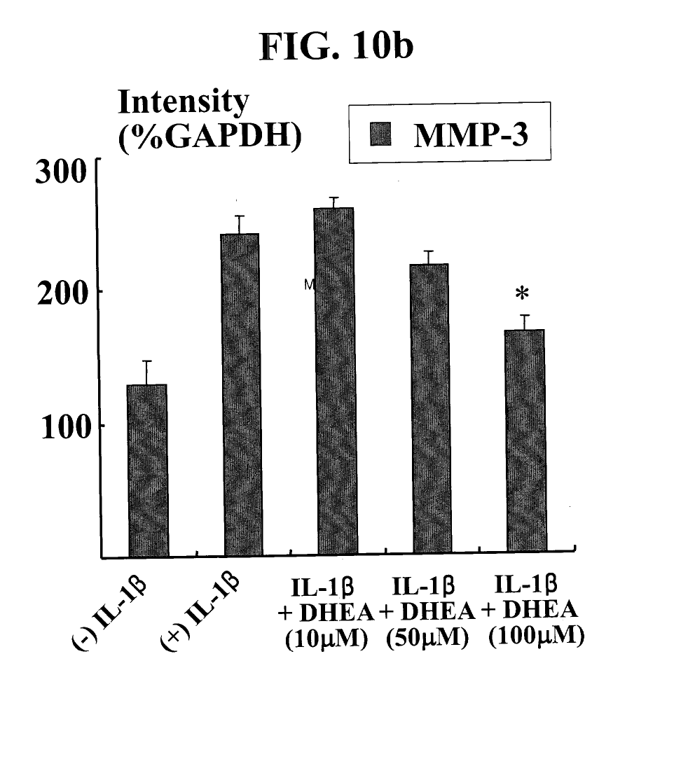

IV. Effects of DHEA on (IL-1β)-Induced MMP-1 and MMP-3 Production (FIGS. 9-10)

To investigate whether DHEA had any suppressive effect on (IL-1β)-induced MMP-1 or MMP-3 expression, chondrocyte cultures were treated with 10, 50, and 100 μM of DHEA together with IL-1β for 72 hr. DHEA suppressed (IL-1β)-induced up-regulation of MMP-1 mRNA in a dose-dependent manner, namely, 74% inhibition with 10 μM and 53% inhibition with 50 μM of DHEA. Inhibitory effect of DHEA on MMP-1 production reached plateau above 50 μM of DHEA. Likewise, DHEA down-regulated the (IL-1β)-induced expression of MMP-3 mRNA at concentration above 10 μM of DHEA in a dose-dependent manner exhibiting 73% inhibition with 100 μM of DHEA.

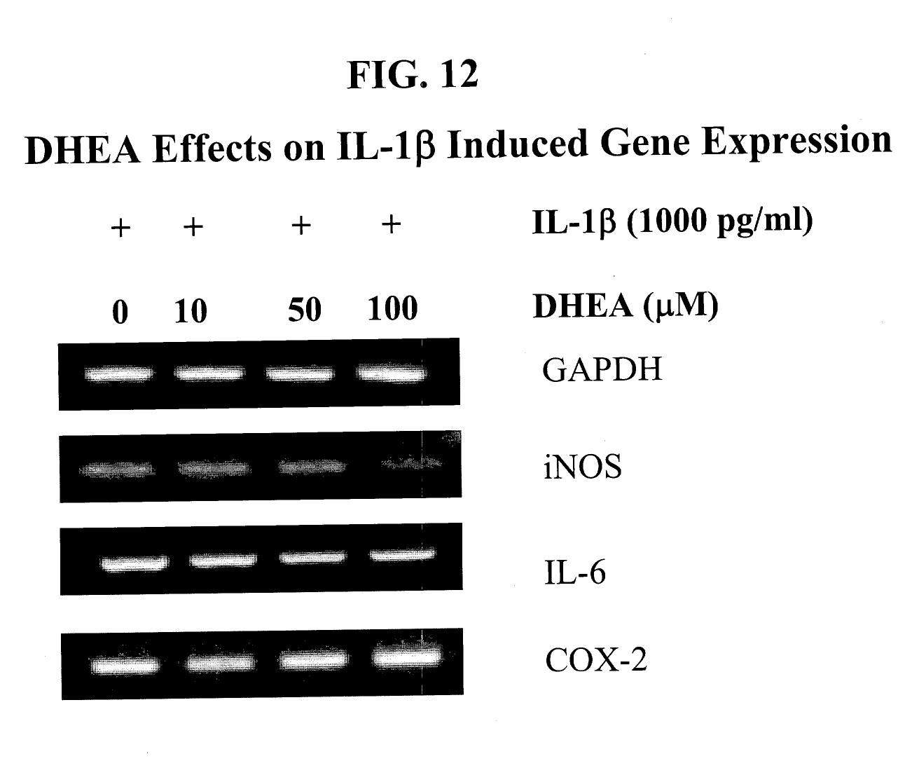

V. Effect of DHEA-Pyruvate on (IL-1β)-Induced iNOS, IL-6 or Cox-2 Gene Expression (FIG. 12)

To investigate whether DHEA had any suppressive effect on (IL-1β)-induced iNOS, IL-6 and Cox-2 expression, chondrocyte was cultured with 10, 50, and 100 μM each of DHEA together with IL-1β for 72 hr. DHEA suppressed (IL-1β)-induced up-regulation of iNOS mRNA in a dose-dependent manner, whereas no significantly change was detectable in IL-6 and Cox-2 gene expression.

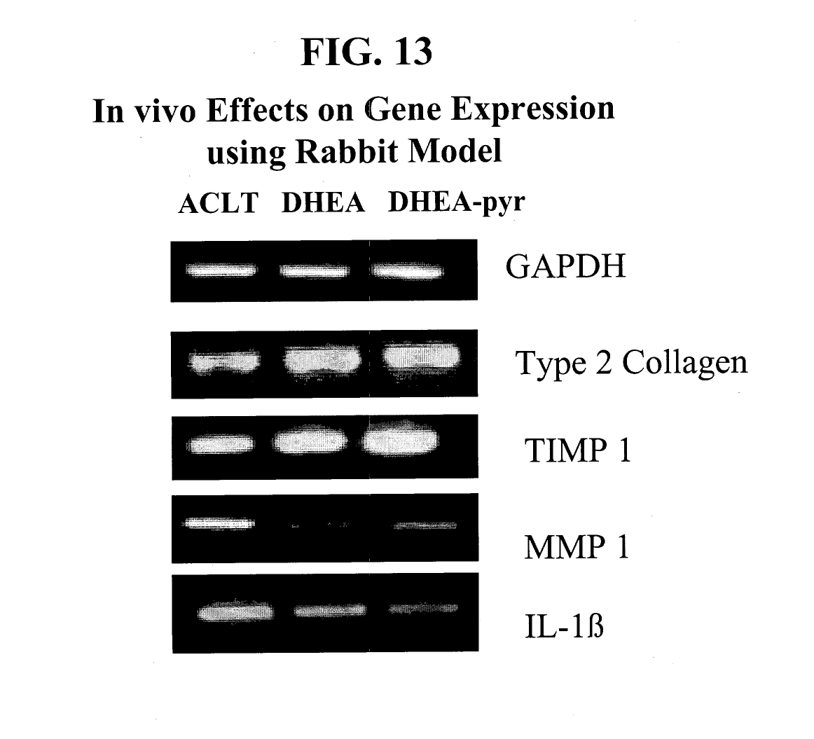

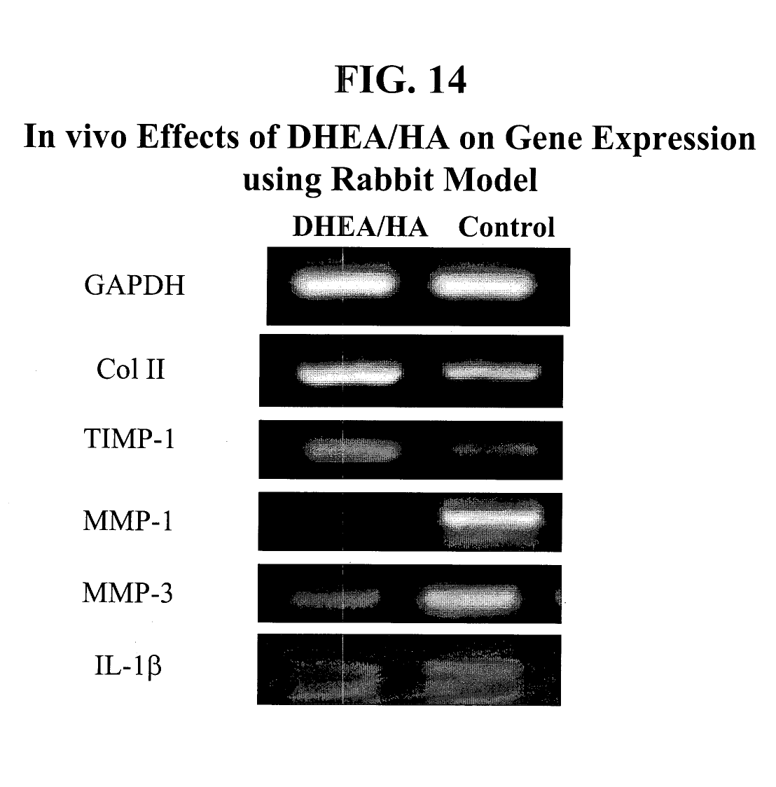

VI. Effects of DHEA and DHEA-Pyruvate on the Articular Cartilage During the Development of Osteoarthritis in Rabbit Knee (FIGS. 13-14)

We investigated the effects of DHEA or DHEA-pyruvate on the expressions of tyep II collagen, TIMP-1, MMP-1 and IL-1β mRNAs in an osteoarthritic rabbit model, in which osteoarthritis had been induced by transection of anterior cruciate ligament (ACLT). In both of DHEA or DHEA-pyruvate treated groups, the expression of tyep II collagen and TIMP-1 was increased.

As shown in FIG. 14, co-administration of both DHEA and HA reduced the expression of MMP-3 significantly while DHEA alone did not change the MMP-3 expression. Therefore, it became obvious that the most preferable composition of this invention comprises a combination DHEA and HA as active ingredients for osteoarthritis treatment.

VII. Topical Application of DHEA to Arthritic Patient

A. Clinical History of Test Subjects

The age of testees ranged from 50 to 71 with an average of 62, consisting of 2 men and 8 women. The disease chronicity was in the range of 2 to 39 years and averaged 5.7 years. Patients' own assessments on arthritic status prior to DHEA treatment are as follows: 3 persons belonged to the category of “exceeding poorness” and 2 to “poorness” in Group I, and 3 to “exceeding poorness”, 1 to “poorness” and 1 to “mediocrity” in Group II.

B. Clinical Assessment

i) Resting Pain

In Group I, the resting pain was averaged 14.34 points before application, scored 15.29 and 15.02 points 1 week and 1 month, respectively, after application, which is considered not a significant change. In Group II, the resting pain was averaged 16.29 points before application, scored 16.14 and 15.33 points 1 week and 1 month, respectively, after application, which is also not a significant change. Therefore, there is no significant increase in pain points between Group I and II.

ii) Motion Pain

In Group I, the motion pain was averaged 8.11 points before application, scored 8.34 and 8.51 points 1 week and 1 month, respectively, after application, which is considered not a significant change. In Group II, the motion pain was averaged 7.37 points before application, scored 7.88 and 8.12 points 1 week and 1 month, respectively after application, which is not a significant change. Therefore, there is no significant increase in pain points between Group I and II.

iii) Pain at Ascending/Descending Stairs

In Group I, the pain at ascending/descending stairs was averaged 10.87 points before application, scored 10.11 and 10.49 points 1 week and 1 month, respectively, after application, which is considered not a significant change. In Group II, the pain at ascending/descending stairs was averaged 11.23 points before application, scored 12.12 and 10.82 points 1 week and 1 month, respectively, after application, which is not a significant change. Therefore, there is no significant increase in pain points between Group I and II.

iv) Joint Effusion

In Group I, the joint effusion was scored 2.12 points before application, 1.98 and 2.27 points 1 week and 1 month, respectively, after application, which is considered not a significant change. In Group II, the joint effusion was scored 2.14 points before application, 2.43 and 2.22 points 1 week and 1 month, respectively, after application, which is no significant increase in pain points between Group I and Group II.

v) Medial Joint Line Tenderness

In Group I, the medial joint line tenderness was scored 2.18 points before application, 2.11 and 2.54 points 1 week and 1 month, respectively, after application, which is considered not a significant change. In Group II, the medial joint line tenderness was scored 2.34 points before application, 2.11 and 2.09 points 1 week and 1 month, respectively, after application.

vi) Opinion of Doctor Regarding the Improvement of Symptoms

In Group I, 1 person belonged to slight improvement and 4 persons to no change, 1 month after application of DHEA. In Group II, the same results as those of Group I were produced.

vii) Opinion of Patient Regarding the Improvement of Symptoms

In Group I, 1 person belonged to slight improvement, 3 persons to no change and 1 person to aggravation 1 month after application of DHEA. In Group II, 1 person belonged to slight improvement and 4 persons to no change.

As described previously, it is understood that the pharmaceutical composition of this invention comprising DHEA or DHEA derivative cannot exert therapeutic effect on arthritis when administered topically. Therefore, summarizing the results in VI and VII, it will be appreciated that the pharmaceutical composition of this invention comprising DHEA or DHEA derivative must be intraarticularly administered into the affected joint to attain effective therapeutic effect on arthritis.

Having described a preferred embodiment of the present invention, it is to be understood that variants and modifications thereof falling within the spirit of the invention may become apparent to those skilled in this art, and the scope of this invention is to be determined by appended claims and their equivalents.

What is claimed is:

1. A pharmaceutical composition for treating IL-1 related disease or disorder, which comprises:
(a) a therapeutically effective dose of dehyroepiandrosterone or its derivative represented by the formula (I); and
(b) a pharmaceutically acceptable carrier: 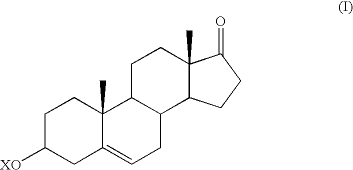
wherein X is H, 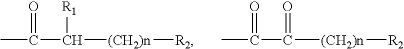
or 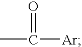
R₁ is H or —NH₂; R₂ is H, —COOH, —NH₂ or 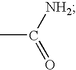
Ar is unsubstituted or substituted phenyl; and n is an integer of 1-20.

2. The pharmaceutical composition according to claim 1, wherein the IL-1 related disease or disorder is selected from the group consisting of (a) inflammatory diseases including osteoarthritis, pancreatitis and asthma; (b) autoimmune diseases including glomerular nephritis, rheumatoid arthritis, scleroderma and alphosis; and (c) infectious diseases including septicemia and septic shock.

3. The pharmaceutical composition according to claim 2, wherein the IL-1 related disease or disorder is osteoarthritis.

4. The pharmaceutical composition according to claim 3, wherein the pharmaceutical composition inhibits the production of matrix metalloproteinase mediated by IL-1.

5. The pharmaceutical composition according to claim 4, wherein the matrix metalloproteinase is matrix metalloproteinase-1 or matrix metalloproteinase-3.

6. The pharmaceutical composition according to claim 5, wherein the matrix metalloproteinase is matrix metalloproteinase-3.

7. The pharmaceutical composition according to claim 3, wherein the pharmaceutical composition promotes the productions of Type II collagen and tissue inhibitor of metalloproteinase.

8. The pharmaceutical composition according to claim 3, wherein the pharmaceutical composition further comprises a hyaluronic acid.

9. The pharmaceutical composition according to any one of claims 3 to 8, wherein the pharmaceutical composition is administered in a manner of intraarticular injection.

10. A method for treating IL-1 related disease or disorder, which comprises administering a patient a pharmaceutical composition comprising (a) a therapeutically effective dose of dehyroisoandrosterone or its derivative represented by the formula (I); and (b) a pharmaceutically acceptable carrier: 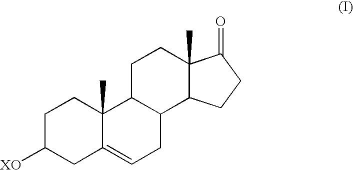
wherein X is H, 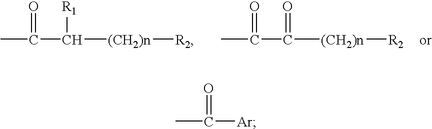
R₁ is H or —NH₂; R₂ is H, —COOH, —NH₂ or 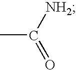
Ar is unsubstituted or substituted phenyl; and n is an integer of 1-20.

11. The method according to claim 10, wherein the IL-1 related disease or disorder is selected from the group consisting of (a) inflammatory diseases including osteoarthritis, pancreatitis and asthma; (b) autoimmune diseases including glomerular nephritis, rheumatoid arthritis, scleroderma and alphosis; and (c) infectious diseases including septicemia and septic shock.

12. The method according to claim 11, wherein the IL-1 related disease or disorder is osteoarthritis.

13. The method according to claim 12, wherein the pharmaceutical composition inhibits the production of matrix metalloproteinase mediated by IL-1.

14. The method according to claim 13, wherein the matrix metalloproteinase is matrix metalloproteinase-1 or matrix metalloproteinase-3.

15. The method according to claim 14, wherein the matrix metalloproteinase is matrix metalloproteinase-3.

16. The method according to claim 12, wherein the pharmaceutical composition promotes the productions of Type II collagen and tissue inhibitor of metalloproteinase.

17. The method according to claim 12, wherein the pharmaceutical composition further comprises a hyaluronic acid.

18. The method according to any one of claims 12 to 17, wherein the administeration is carried out in a manner of intraarticular injection.
